(19) United States
(12) Patent Application Publication
(10) Pub. No.: US 2004/0098059 A1
(43) Pub. Date: May 20, 2004

(54) Method and apparatus for termination of cardiac tachyarrhythmias

(75) Inventors: Victor T. Chen, Minnetrista, MN (US); Stephen J. Hahn, Shoreview, MN (US)

Correspondence Address: SCHWEGMAN, LUNDBERG, WOESSNER & KLUTH, P.A., P.O. BOX 2938, MINNEAPOLIS, MN, 55402, US

(73) Assignee: Cardiac Pacemakers, Inc.

(21) Appl. No.: 10/629,343

(22) Filed: Jul. 28, 2003

Related U.S. Application Data
(63) Continuation of application No. 09/448,648, filed on Nov. 24, 1999, now abandoned.

(51) Int. Cl. A61N 1/362

(52) U.S. Cl. 607/14

(57) ABSTRACT

An apparatus and method for terminating atrial and ventricular tachyarrhythmias by delivering a voltage pulse to an electrode arrangement that efficiently terminates the tachyarrhythmia. In one embodiment, a voltage pulse is impressed between an electrode located in the coronary sinus and an electrode located within the superior vena cava or right atrium which is also coupled to an extravascular electrode. In another embodiment, a voltage pulse is impressed between an electrode located within the right ventricle and electrodes located within the coronary sinus and the superior vena cava that are also coupled to an extravascular electrode.

### CROSS-REFERENCE TO RELATED APPLICATION(S)

This application is a continuation of U.S. patent application Ser. No. 09/448,648, filed on Nov. 24, 1999, the specification of which is incorporated herein by reference.

### FIELD OF THE INVENTION

This invention pertains to apparatus and methods for treating cardiac arrhythmias. In particular, the invention relates to an apparatus and method for electrically terminating tachyarrhythmias.

### BACKGROUND

Tachyarrhythmias are abnormal heart rhythms characterized by a rapid heart rate. Examples of tachyarrhythmias include supraventricular tachycardias such as sinus tachycardia, atrial tachycardia, and atrial fibrillation (AF), and ventricular tachyarrhythmias such as ventricular tachycardia (VT) and ventricular fibrillation (VF). Both ventricular tachycardia and ventricular fibrillation are hemodynamically compromising, and both can be life-threatening. Ventricular fibrillation, however, causes circulatory arrest within seconds and is the most common cause of sudden cardiac death. Atrial fibrillation is not immediately life threatening, but since atrial contraction is lost, the ventricles are not filled to capacity before systole which reduces cardiac output. This may cause lightheadedness or fainting in some individuals, as well as fatigue and shortness of breath, hindering the individual from carrying out normal daily activities. If atrial fibrillation remains untreated for long periods of time, it can also cause blood to clot in the left atrium, possibly forming an emboli and placing patients at risk for stroke.

Cardioversion (an electrical shock delivered to the heart synchronously with an intrinsic depolarization) and defibrillation (an electrical shock delivered without such synchronization) can be used to terminate most tachycardias, including AF, VT, and VF. As used herein, the term defibrillation should be taken to mean an electrical shock delivered either synchronously or not in order to terminate a fibrillation. In electrical defibrillation, a current depolarizes a critical mass of myocardial cells so that the remaining myocardial cells are not sufficient to sustain the fibrillation. The electric shock may thus terminate the tachyarrhythmia by depolarizing excitable myocardium, which prolongs refractoriness, interrupts reentrant circuits, and discharges excitatory foci.

Implantable cardioverter/defibrillators (ICDs) provide electro-therapy by delivering a shock pulse to the heart when fibrillation is detected by the device. The ICD is a computerized device containing a pulse generator that is usually implanted into the chest or abdominal wall. Electrodes connected by leads to the ICD are placed on the heart, or passed transvenously into the heart, to sense cardiac activity and to conduct the impulses from the pulse generator. Typically, the leads have electrically conductive coils along their length that act as electrodes. ICDs can be designed to treat either atrial or ventricular tachyarrhythmias, or both, by delivering a shock pulse that impresses an electric field between the electrodes to which the pulse generator terminals are connected. The electric field vector applied to the heart is determined by the magnitude of the voltage pulse and the physical arrangement of the shocking electrodes, which may serve to concentrate the field in a particular region of the heart. Thus, the particular electrode arrangement used will dictate how much depolarizing current is necessary in order to terminate a given tachyarrhythmia.

Ventricular and atrial fibrillation are probabilistic phenomena that observe a dose-response relationship with respect to shock strength. The ventricular defibrillation threshold (VDFT) is the smallest amount of energy that can be delivered to the heart to reliably revert ventricular fibrillation to normal sinus rhythm. Similarly, the atrial defibrillation threshold (ADFT) is the threshold amount of energy that will terminate an atrial fibrillation. Electrical energy delivered to the heart has the potential to both cause myocardial injury and subject the patient to pain. Whether or not a particular patient is a suitable candidate for ICD implantation is determined in part by that patient's defibrillation threshold, since too a high a threshold would necessitate electrical shock therapy at levels that are dangerous for the patient. Furthermore, the larger the magnitude of the shocks delivered by an ICD, the more the battery is drained, thus decreasing the longevity of the device. It is desirable, therefore, for the defibrillation threshold to be as small as possible in order to minimize the amount of shocking current that the ICD must deliver in order to terminate a given tachyarrhythmia.

Electrode arrangements have been devised in an attempt to minimize the defibrillation threshold for particular types of tachyarrhythmias. For example, the traditional configuration for ventricular defibrillation is to place a cathodic electrode in the right ventricle, with the anode formed jointly by an electrode placed in the superior vena cava and the conductive housing of the ICD acting as an additional electrode. For treating atrial fibrillation, a conventional electrode configuration is to use electrodes disposed within the coronary sinus and in the right atrium. A further modification to the configuration that has been suggested by some investigators is to electrically connect an electrode placed in the right ventricle in common with the coronary sinus electrode.

In order to further improve safety and avoid unnecessary discomfort for ICD patients, there is a continuing need for methods and apparatus that reduce the defibrillation threshold. Such reductions in defibrillation thresholds may also expand the population of patients for whom ICDs are an appropriate therapeutic option. It is toward this general objective that the present invention is directed.

### SUMMARY OF THE INVENTION

The present invention is a method and apparatus for terminating tachyarrhythmias such as fibrillation by the efficient delivery of electrical energy through an electrode configuration to the heart in response to sensed electrical events from a sensing channel that indicate the occurrence of a tachyarrhythmia. In one embodiment of the invention, the defibrillation energy is imparted to the heart by a pulse generator having one terminal connected to a first electrode disposed within the coronary sinus and another terminal connected to a second electrode disposed within the superior vena cava or right atrium and to an extravascular electrode located in proximity to the heart. In another embodiment, the pulse generator has one terminal connected to a first electrode disposed within the right ventricle and another terminal connected a second electrode disposed within the superior vena cava or right atrium, a third electrode disposed within the coronary sinus, and an extravascular electrode. The extravascular electrode may be a cutaneous patch or may be the conductive housing of the apparatus. The voltage pulse of the pulse generator may be monophasic in which the electrode connected to one of the terminals is a cathode and the electrode connected to the other terminal forms an anode, or may be biphasic in which the polarity of the pulse generator terminals alternates during the pulse.

### BRIEF DESCRIPTION OF THE DRAWINGS

FIG. 1 is a system diagram of an apparatus for terminating tachyarrhythmias with electrical energy.

FIG. 2 shows an electrode configuration in accordance with one embodiment of the invention.

FIG. 3 shows an electrode configuration in accordance with another embodiment of the invention.

### DESCRIPTION OF PARTICULAR EMBODIMENTS

In the description of particular embodiments that follows, a microprocessor-based ICD will be referred to as incorporating the system and method that is the present invention where programmed instructions in memory are executed by a microprocessor. It should be appreciated, however, that certain functions of an ICD can be controlled by custom logic circuitry either in addition to or instead of a programmed microprocessor. The term “circuitry” as used herein should therefore be taken to mean either custom circuitry (i.e., dedicated hardware) or a microprocessor executing programmed instructions contained in a processor-readable storage medium along with associated circuit elements.

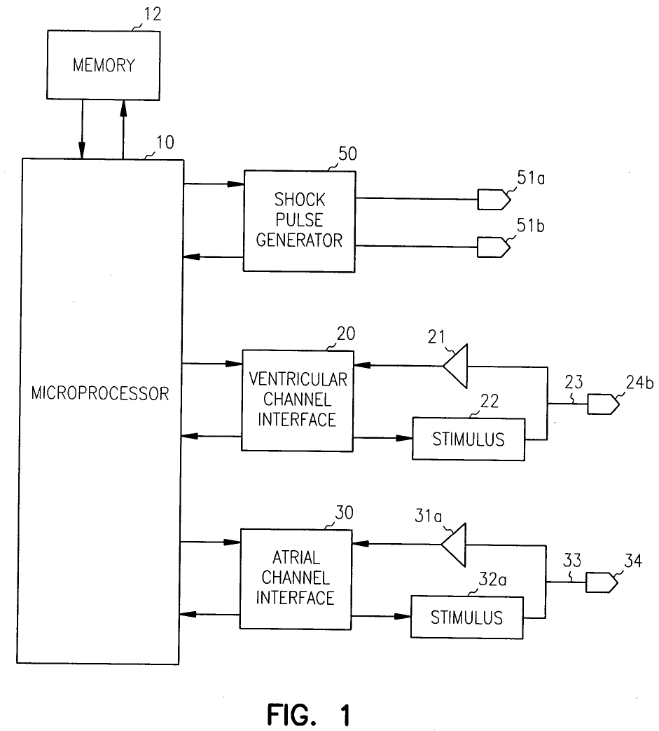

FIG. 1 is a system diagram of a microprocessor-based implantable cardioverter/defibrillator with the capability of also delivering pacing therapy. A microprocessor 10 communicates with a memory 12 via a bidirectional data bus. The memory 12 typically comprises a ROM for program storage and a RAM for data storage. The ICD has atrial sensing and pacing channels comprising electrode 34, lead 33, sensing amplifier 31, pulse generator 32, and an atrial channel interface 30 which communicates bidirectionally with a port of microprocessor 10. The ventricular sensing and pacing channels similarly comprise electrode 24, lead 23, sensing amplifier 21, pulse generator 22, and a ventricular channel interface 20. For each channel, the same lead and electrode are used for both sensing and pacing. The sensing channels are used to control pacing and for measuring heart rate in order to detect tachyarrythmias such as fibrillation. The ICD detects a ventricular tachyarrhythmia, for example, by measuring a heart rate via the ventricular sensing channel and determining whether the rate exceeds a selected threshold value. A shock pulse generator 50 is also interfaced to the microprocessor for delivering cardioversion or defibrillation pulses to the heart via a pair of terminals 51a and 51b that are connected by defibrillation leads to shock electrodes placed in proximity to regions of the heart. The defibrillation leads have along their length electrically conductive coils that act as electrodes for defibrillation stimuli. The defibrillation leads and electrodes used in any of the described embodiments below may be implemented as lead-body electrodes that are either single elongated coils or made up of a plurality of smaller bands. The delivered voltage pulses may be either monophasic or biphasic. The shock pulse generator as well as the rest of the circuitry are powered by a battery power supply. The device is enclosed by a housing which may be implanted by placing it an abdominal wall pocket, or preferably, in a pectoral pocket either subcutaneously or under the pectoralis major muscle. The leads from the housing are advanced to the heart transvenously, with venous access through the cephalic or subclavian veins. The defibrillation leads are connected to one of the pulse generator terminals.

In one primary embodiment of the invention, an electrode configuration is used which is particularly suited for terminating atrial arrhythmias. In this configuration, a lead with a first distal shocking electrode is situated in the coronary sinus (CS) such that the electrode resides in the left lateral heart, just beneath the atrial appendage. The first electrode is connected to one terminal of the pulse generator so as to act as a cathode during a monophasic voltage pulse. A second shocking electrode is connected through its lead to another terminal of the pulse generator so as to form an anode during the voltage pulse and is disposed within the superior vena cava (SVC). Also connected to the pulse generator terminal in common with the second electrode so as to also form an anode is the conductive housing of the device (also referred to as the cannister or CAN). The polarity of the arrangement during a monophasic pulse is thus designated as:

CS⁻→SVC⁺+CAN⁺

with the CS electrode acting as the sole cathode and the SVC and CAN electrodes acting as joint anodes for the monophasic defibrillation stimulus.

In another primary embodiment, an electrode configuration is used that is particularly suited for ventricular defibrillation. In this arrangement, a lead with a first distal shocking electrode is situated in the right ventricle, with the first electrode connected to one terminal of the pulse generator so as to act as a cathode during a monophasic voltage pulse. Second and third shocking electrodes are connected through their respective leads to the other terminal of the pulse generator so as to form a joint anode during the voltage pulse and are disposed within the superior vena cava (SVC) and coronary sinus (CS), respectively. Also connected to the pulse generator terminal in common with the second and third electrodes so as to also form a joint anode is the conductive housing of the device. The polarity of the arrangement during a monophasic pulse is thus designated as:

RV⁻→CS⁺+SCV⁺+CAN⁺

with the RV electrode as the sole cathode and the CS, SVC, and CAN electrodes acting as joint anodes for a monophasic defibrillation pulse.

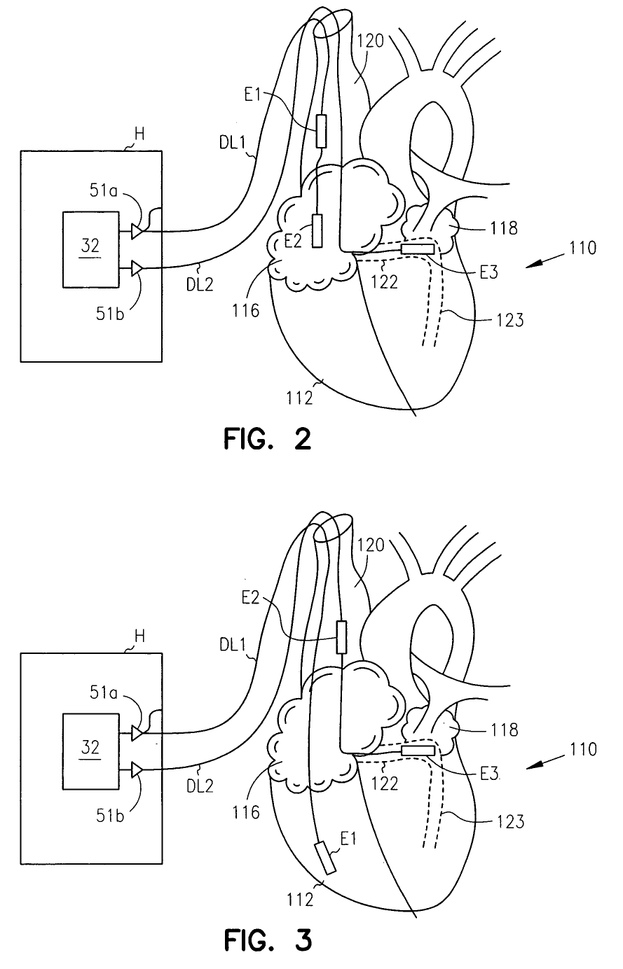

FIGS. 2 and 3 illustrate the configurations just described. Both figures show a heart 10, the superior vena cava 120, right atrium 116, right ventricle 112, coronary sinus 122, cardiac vein 123, and left atrium 118. FIG. 2 shows a configuration corresponding to the first primary embodiment in which the terminals 51a and 51b of the pulse generator 32 are connected to defibrillation leads DL1 and DL2, respectively. Defibrillation lead DL1 is connected to electrodes E1 and E2 which are situated in the superior vena cava 120 and right atrium 116, respectively. Defibrillation lead DL2 is connected to electrode E3 which is disposed within the coronary sinus 122. Terminal 51a is also electrically connected to the device housing H so that the housing forms an extravascular electrode electrically in common with electrodes E1 and E2.

FIG. 3 shows a configuration corresponding the second primary embodiment described above. Terminal 51a of pulse generator 32 is connected to the housing H so as to form an extravascular electrode and to defibrillation lead DL1. The lead DL1 is connected to electrode E2 situated in the superior vena cava 120 to electrode E3 disposed within the coronary sinus 122. The housing H, electrode E2, and electrode E3 thus form a joint electrode. Terminal 51b is connected to lead DL2 which is connected to electrode E1 which is located in the right ventricle 112.

The embodiments described above may be modified to form further exemplary embodiments as follows. First, the polarity of the monophasic defibrillation pulse may be reversed so that the first-described embodiment becomes:

CS⁺→SVC⁻+CAN⁻

and the second-described embodiment becomes:

RV^{→+}CS⁻+SCV⁻+CAN⁻

In a preferred embodiment, however, a biphasic defibrillation pulse is employed in which the polarity of the pulse generator alternates during the pulse.

In the primary embodiments described above, the conductive housing was used as an extravascular electrode placed in proximity to the heart and connected to one of the pulse generator terminals. In a modified embodiment, an additional subcutaneous array electrode (SQA) maybe employed which is located, for example, in the left maxillary space and which is connected so as to be electrically common with the housing. Thus, the polarity would be designated in the case of the atrial defibrillation embodiment as:

CS⁻→SVC⁺+CAN⁺+SQA⁺

and for the ventricular defibrillation embodiment as:

RV⁻→CS⁺+SCV⁺+CAN⁺+SQA⁺

In another embodiment, the housing electrode is replaced by the subcutaneous array electrode which is then the sole extravascular electrode. In an implementation of either of the primary embodiments as described in which the device is to be used externally rather than being implanted, the housing electrode is replaced by a cutaneous patch electrode.

In another modification to the described embodiments, the SVC electrode is replaced by an electrode in the right atrium (RA) or situated in the right atrial appendage (RAA), which electrodes may be formed along the length of the catheter or not. In further modifications, the SVC or RA electrode may be situated such that it lies partly within the SVC and partly within the RA, or the SVC electrode may extend to the innominate vein. A combination of an RA and SVC electrodes connected electrically in common may also be used, with the RA and SVC electrodes on the same or different lead bodies.

Although the invention has been described in conjunction with the foregoing specific embodiment, many alternatives, variations, and modifications will be apparent to those of ordinary skill in the art. Such alternatives, variations, and modifications are intended to fall within the scope of the following appended claims.

What is claimed is:

1. An apparatus for delivering electrical energy to a heart in order to terminate a tachyarrhythmia, comprising:
a sensing channel for detecting electrical events in the heart and producing sensing signals in accordance therewith;
processing circuitry for detecting the occurrence of a tachyarrhythmia from the sensing signals;
an electrode arrangement consisting of a first electrode for disposition within the coronary sinus, a second electrode for disposition within the superior vena cava or right atrium, and an extravascular electrode for location in proximity to the heart; and,
a pulse generator for delivering a voltage pulse between first and second terminals, wherein the first terminal is connected to the first electrode, and the second terminal is connected to the second and extravascular electrodes.

2. The apparatus of claim 1 wherein the first and second terminals of the pulse generator are made electrically positive and negative, respectively, during delivery of a voltage pulse.

3. The apparatus of claim 1 wherein the pulse generator delivers a biphasic voltage pulse.

4. The apparatus of claim 1 wherein the extravascular electrode is a cutaneous patch.

5. The apparatus of claim 1 wherein the extravascular electrode is an implantable housing.

6. An apparatus for delivering electrical energy to a heart in order to terminate a tachyarrhythmia, comprising:
a sensing channel for detecting electrical events in the heart and producing sensing signals in accordance therewith;
processing circuitry for detecting the occurrence of a tachyarrhythmia from the sensing signals;
a first electrode for disposition within the right ventricle, a second electrode for disposition within the superior vena cava or right atrium, a third electrode for disposition within the coronary sinus, and an extravascular electrode for location in proximity to the heart; and,
a pulse generator for delivering a voltage pulse between first and second terminals, wherein the first terminal is connected to the first electrode, and the second terminal is connected to the second, third, and extravascular electrodes.

7. The apparatus of claim 6 wherein the first and second terminals of the pulse generator are made electrically positive and negative, respectively, during delivery of a voltage pulse.

8. The apparatus of claim 6 wherein the pulse generator delivers a biphasic voltage pulse.

9. The apparatus of claim 6 wherein the extravascular electrode is a cutaneous patch.

10. The apparatus of claim 6 wherein the extravascular electrode is an implantable housing.

11. A method for terminating an atrial tachyarrhythmia occurring in a patient's heart, comprising:
constructing an electrode arrangement consisting of a first electrode disposed within the coronary sinus, a second electrode disposed within the superior vena cava or right atrium, and an extravascular electrode located in proximity to the heart;
detecting the occurrence of an atrial tachyarrhythmia;
delivering a voltage pulse to first and second terminals wherein the first terminal is connected to the first electrode, and the second terminal is connected to the second and extravascular electrodes.

12. The method of claim 11 wherein the first and second terminals of the pulse generator are made electrically positive and negative, respectively, during delivery of a voltage pulse.

13. The method of claim 11 wherein the pulse generator delivers a biphasic voltage pulse.

14. The method of claim 11 wherein the extravascular electrode is a cutaneous patch.

15. The method of claim 11 wherein the extravascular electrode is an implantable housing.

16. A method for terminating a ventricular tachyarrhythmia occurring in a patient's heart, comprising:
disposing a first electrode within the right ventricle, a second electrode within the superior vena cava or right atrium, a third electrode within the coronary sinus, and an extravascular electrode in proximity to the heart;
detecting the occurrence of a ventricular tachyarrhythmia; and,
delivering a voltage pulse to first and second terminals wherein the first terminal is connected to the first electrode, and the second terminal is connected to the second, third, and extravascular electrodes.

17. The method of claim 16 wherein the first and second terminals of the pulse generator are made electrically positive and negative, respectively, during delivery of a voltage pulse.

18. The method of claim 16 wherein the pulse generator delivers a biphasic voltage pulse.

19. The method of claim 16 wherein the extravascular electrode is a cutaneous patch.

20. The method of claim 16 wherein the extravascular electrode is an implantable housing.
